(19) United States
(12) Patent Application Publication
(10) Pub. No.: US 2010/0331361 A1
(43) Pub. Date: Dec. 30, 2010

(54) PHARMACEUTICAL COMPOSITION CONTAINING ALPHA-ADRENERGIC RECEPTOR ANTAGONIST AND AN ANTI-MUSCARINIC AGENT AND METHOD OF IMPROVING LOWER URINARY TRACT SYMPTOMS ASSOCIATED WITH PROSTATIC HYPERTROPHY

(75) Inventors: Hidehiro Kakizaki, Hokkaido (JP); Masaki Yoshida, Kumamoto (JP); Takeshi Uchida, Tokyo (JP); Karin Juliette van Charldorp, Leiderdorp (NL); Brigitte Johanna Fanny Bosman, Leiderdorp (NL); Monique Maria Alida Klaver, Leiderdorp (NL); Alberto Garcia Hernandez, Leiderdorp (NL); Tennis Edwin Drogendijk, Leiderdorp (NL)

Correspondence Address: FITZPATRICK CELLA HARPER & SCINTO, 1290 Avenue of the Americas, NEW YORK, NY, 10104-3800, US

(73) Assignees: ASTELLAS PHARMA INC., Tokyo (JP); ASTELLAS IRELAND CO., LTD., Mulhuddart (IE)

(21) Appl. No.: 12/688,426

(22) Filed: Jan. 15, 2010

Related U.S. Application Data
(63) Continuation-in-part of application No. PCT/JP2007/074717, filed on Dec. 21, 2007.
(60) Provisional application No. 60/929,996, filed on Jul. 20, 2007.

(51) Int. Cl. A61K 31/4725; A61P 13/02

(52) U.S. Cl. 514/305

(57) ABSTRACT

A pharmaceutical composition including active ingredients of (R)-5-(2-{[2-(2-ethoxyphenoxy)ethyl]amino}propyl-2-methoxybenzene-1-sulfonamide and (1S)-1-phenyl-1,2,3,4-tetrahydroisoquinoline-2-carboxylic acid (3R)-quinuclidin-3-yl ester, or their pharmaceutically acceptable salts, provides improvement of lower urinary tract symptoms associated with prostatic hypertrophy. The active ingredients may be administered either simultaneously or at a time interval. The pharmaceutical composition also provides significant improvement of lower urinary tract symptoms associated with benign prostatic hyperplasia having a substantial storage component.

### BACKGROUND OF THE INVENTION

1. Field of the Invention

The present invention relates to a pharmaceutical composition containing tamsulosin, or its pharmaceutically acceptable salt, and solifenacin, or its pharmaceutically acceptable salt, as active ingredients and its use as an agent to improve lower urinary tract symptoms associated with prostatic hypertrophy, especially those having a substantial storage component.

2. Brief Description of the Background Art

The present invention relates to a pharmaceutical composition comprising an α-adrenergic receptor antagonist and an anti-muscarinic agent which is useful for improvement of lower urinary tract symptoms (hereinafter sometimes referred to as “LUTS”) associated with prostatic hypertrophy, especially benign prostatic hyperplasia (hereinafter sometimes referred to as “BPH”). The α-adrenergic receptor antagonist and anti-muscarinic agent may be administered together or separately at an interval.

In particular, the α-adrenergic receptor antagonist and an anti-muscarinic agent, preferably, respectively contain as active ingredients (R)-5-(2-{[2-(2-ethoxyphenoxy)ethyl]amino}propyl-2-methoxybenzene-1-sulfonamide (hereinafter sometimes referred to as tamsulosin) or its pharmaceutically acceptable salt, and (1S)-1-phenyl-1,2,3,4-tetrahydroisoquinoline-2-carboxylic acid (3R)-quinuclidin-3-yl ester (hereinafter sometimes referred to as solifenacin) or its pharmaceutically acceptable salt.

The present invention also relates to use of α-adrenergic receptor antagonist and an anti-muscarinic agent, and especially tamsulosin and/or solifenacin and/or their pharmaceutically acceptable salts for the preparation of a medicament for the treatment of lower urinary tract symptoms associated with benign prostatic hyperplasia having a substantial storage component

The prostate gland is a male organ surrounding the urethra between the neck of the bladder and the external urethral sphincter muscle. Prostatic adenoma observed in prostatic hypertrophy is a benign tumor originating from the transitional zone (internal gland) of the prostate, and its growth depends on male hormones (androgens), but its detailed etiological mechanisms have yet to be clarified.

Lower urinary tract symptoms associated with benign prostatic hyperplasia or (hereinafter sometimes referred to as “LUTS/BPH”), previously referred to in the art as symptomatic BPH, is a common condition in men over the age of 50 years. It occurs in approximately 20% of men younger than 65 years and in 40% of men more than 65 years of age (Br. J. Gen. Pract., Vol. 43 (1993) 318-21e).

Development of prostatic hypertrophy is considered to arise mainly from direct urethral compression (mechanical occlusion) due to hypertrophy of the adenoma and from an intra-urethral pressure increase (functional occlusion) due to excessive contraction of the prostate and urethra via sympathetic nerves, and the like. The incidence of histologically defined prostatic hypertrophy increases with age, and has been reported to be observed in 90% of 70-80 year-old men.

LUTS includes storage symptoms (such as frequency, urgency and nocturia) as well as voiding symptoms (such as hesitancy, weak stream, intermittency, incomplete bladder emptying, dribbling and abdominal straining). Although voiding symptoms are more prevalent than storage symptoms, storage symptoms are considered to be the most bothersome. Storage symptoms also interfere to the greatest extent with daily life activities and have a major impact on quality of life (J. Urol., Vol. 157 (1997) 885-89).

BPH refers to a histological diagnosis: a process characterised by stromal and epithelial cell hyperplasia beginning in the periurethral zone of the prostrate. Although nearly all men develop histological BPH, the degree of prostatic enlargement resulting from hyperplasia is highly variable. Its etiology is not fully understood, but both age and male testosterone levels contribute to its progression.

LUTS associated with BPH may be due to obstruction caused by an increased prostatic size induced by an increase in the number of prostatic cells. It may also be due to obstruction caused by an increased contraction of smooth muscle cells in the prostate, urethra and bladder neck (Br. J. Urol., Vol. 47 (1975) 193-202). However the relationship between prostatic enlargement and LUTS is not very strong (JAMA, Vol. 270, No. 7 (1993) 860-64; Urology, Vol. 58, No. 6, Supp. 1 (2001) 5-16). There is increasing evidence that in addition to obstruction at the level of the prostrate, other factors such as detrusor disorders, central nervous system disorders, ageing and/or ischemia may contribute to the development of LUTS associated with BPH (Eur. Urol., Vol. 40, Supp. 4 (2001) 1-4).

The storage symptoms observed in males with LUTS associated with BPH may be due to co-existing detrusor overactivity, as the occurrence of both conditions increases with age, and could be secondary to bladder outlet obstruction. Bladder outlet obstruction could lead to cholinergic denervation of the detrusor and consequent supersensitivity of muscarinic receptors to acetylcholine. Increased bladder outlet resistance may also result in ischemia, increased detrusor collagen content, changes in electrical properties of detrusor smooth muscle cells and reorganisation of the spinal micturition reflex, all of which are associated with the development of detrusor overactivity in animal models (Eur. Urol., Vol. 49 (2006) 651-59).

The prevalence of detrusor overactivity in men with LUTS associated with BPH has been estimated to be between 40 and 70% (J. Urol., Vol. 66 (2001) 550; Scand. J. Urol. Nephrol., Vol. 35 (2001) 463). If left untreated, LUTS can worsen and in the long term may eventually lead to (irreversible) bladder dysfunction and an increased risk of serious complications such as acute urinary retention (hereinafter sometimes referred to as “AUR”) (Eur. Urol., Vol. 39 (2001) 390-99).

Dysuria associated with prostatic hypertrophy is considered to arise from the above-mentioned hypertrophy of the prostate as well as from excessive contraction of the prostate and urethra, and appears as a wide variety of lower urinary tract symptoms such as voiding symptoms (weakened stream, interrupted stream, delayed urination, abdominal pressure urination, and the like), storage symptoms (pollakiuria, nocturia, urgency, and the like), and post-voiding symptoms (emptying, dribbling after urination, and the like). The severity of prostatic hypertrophy is evaluated by various criteria such as urinary flow-metric tests, prostate weight, and the International Prostatic Symptom Scoring (I-PSS).

I-PSS is one of the criteria to evaluate the severity of the foregoing lower urinary tract symptoms by scoring responses to the following questions:

(1) During the last month, have you had a sensation of residual urine after urinating?
(2) During the last month, have you had to urinate again in less than two hours after you last urinated?
(3) During the last month, have you experienced interrupted urination?
(4) During the last month, have you found it difficult to postpone urination?
(5) During the last month, have you had a weak urinary stream?
(6) During the last month, have you had to strain to urinate?
(7) During the last month, how many times did you typically have to get up to urinate after you went to bed until you got up in the morning?

The indices (1), (3), (5) and (6) evaluate voiding symptoms, and indices (2), (4) and (7) evaluate storage symptoms. Among these, responses to (1)-(6) are scored: 0 if the response was not at all, 1 if fewer than 1 time in 5, 2 if fewer than 1 time in 2, 3 if about 1 time in 2, 4 if more than 1 time in 2, and 5 if almost always. Additionally, the responses to (7) are respectively scored: 0, 1, 2, 3, 4, and 5 if the response was none, 1 time, 2 times, 3 times, 4 times, and 5 times or more.

Patients whose total I-PSS scores are 8 or higher are diagnosed as severe enough to be treated for prostatic hypertrophy. Patients whose total I-PSS scores are 13 or higher are diagnosed as severe enough to be treated for lower urinary tract symptoms associated with benign prostatic hyperplasia in accordance with the method of the present invention. These latter patients are deemed to present a substantial storage component and so benefit from the combined use of α-andrenergic receptor antagonist and anti-muscarinic agent.

Medical treatment is first-line therapy for LUTS associated with BPH. As representative therapeutic drugs currently in use for prostatic hyperplasia, adrenaline α receptor antagonists aimed at ameliorating the functional occlusion are known and used clinically. The mechanism of action of the adrenaline α receptor antagonists is based on the notion that the receptor involved in the contraction of the prostate and urethral smooth muscle is the adrenaline α receptor. Tamsulosin is an example of such compound, which mainly targets the prostate. As a consequence the strongest symptomatic improvements as measured with the I-PSS are seen in the voiding symptoms, in particular improvement of weak stream.

As representative therapeutic drugs currently in use for prostatic hypertrophy, adrenaline α₁ receptor antagonists aimed to ameliorate the functional occlusion are widely known and being used clinically as first choice drugs for treating dysuria associated with prostatic hypertrophy. The mechanism of action of the adrenaline α₁ receptor antagonists is based on the notion that the receptor involved in the contraction of the prostate and urethral smooth muscle is the adrenaline α₁ receptor.

On the other hand, the prostate is a target organ of male hormones, androgens, which are intimately involved in the genesis and development of prostatic hypertrophy (hyperplasia). As drugs to inhibit such androgens action, chlormadinone acetate and allylestrenol are used. Additionally, finasteride and dutasteride, which are drugs that inhibit the enzyme (5α reductase) that converts testosterone to dihydrotestosterone in the prostate, are used for treatment in order to improve dysuria or LUTS associated with prostatic diminution.

In view of its mechanism of action, a muscarinic receptor antagonist may be effective in improving storage symptoms, but it could antagonize the improving effect on voiding symptoms, since it may inhibit contraction of the bladder at the time of urination. Actually, when improvement of lower urinary tract symptoms associated with prostatic hyperplasia is desired, a muscarinic receptor antagonist should be administered with great caution—or is even contra-indicated—because it may cause urinary retention or anuresis that may necessitate catheterization or surgical intervention (Br. J. Urol., Vol. 47, No. 2 (1975) 193-202; Folia Pharmacologica Japonica, Vol. 12 (2003) 331).

For example, in Japan, an insert to solifenacin succinate, a muscarinic receptor antagonist, states that it is contra-indicated “in patients with anuresis” because “it may inhibit contraction of the bladder at the time of urination and thus deteriorate the symptom”, and that “in patients with diseases accompanied with lower urinary tract occlusion (prostatic hyperplasia, and the like.),” it should be administered with great caution because “it may cause anuresis by its anti-cholinergic action”. The insert also gives an important basic warning that “In patients with dysuria (lower urinary tract occlusion diseases [prostatic hyperplasia, and the like] or voiding muscle contraction disturbances, and the like), residual urine volume should be measured before administration of this drug and special tests should be considered when necessary. Also, the patients should be kept under careful observation throughout the administration period by periodically confirming absence of aggravation of dysuria.”

However, in spite of the fact that a muscarinic receptor antagonist may worsen voiding symptoms due to its own mechanism of action, the combined use of an alpha-adrenergic receptor antagonist and an anti-muscarinic agent for the treatment of LUTS/BPH has been advocated in EP-1123707. A long list of alpha-adrenergic receptor antagonists and anti-muscarinic agents had been disclosed, but the most preferred combinations were doxazosin and darifenacin, and 4-amino-6,7-dimethoxy-2-(5-methanesulfonamido-1,2,3,4-tetrahydroisoquinol-2-yl)-5-(2-pyridyl)quinazoline (also known as UK 338,003) and darifenacin. Although the efficacy of the combinations was said to be assessed on the basis of the I-PSS-questionnaire no description of the results of any clinical study was provided. J. Urol., Vol. 174 (2005) 1334-38 described the results of a clinical study in male patients having a history of: over six months of proven lower urinary tract symptoms associated with prostatic hyperplasia, having at least one urgency episode per day, having eight or more micturitions per day, treated with the combination of the α-adrenergic receptor antagonist doxazosin, and the muscarinic receptor antagonist propiverine hydrochloride.

To date, several combination therapies utilizing an adrenaline α receptor antagonist and a muscarinic receptor antagonist have been known to improve lower urinary tract symptoms associated with prostatic hypertrophy, but no combination of an adrenaline α receptor antagonist and a muscarinic receptor antagonist have been known to be equal or superior to therapies with an adrenaline α receptor alone in efficacy to improve voiding symptoms in terms of I-PSS. Indeed, in view of the possibility that a muscarinic receptor antagonist may worsen voiding symptoms owing to its own mechanism of action, one of ordinary skill in the art would expect that, while the combination therapy with the adrenaline α receptor antagonist and the muscarinic receptor antagonist further improved storage symptoms compared to the therapy with the adrenaline α receptor alone, the combination therapy would antagonize the improving effect in voiding symptoms, namely, it would exert a negative effect on voiding symptoms.

For example, there is a report in the literature which described efficacy of a therapy using a combination of doxazosin, an adrenaline α receptor antagonist, and propiverine hydrochloride, a muscarinic receptor antagonist, on lower urinary tract symptoms associated with prostatic hypertrophy (J. Urology, Vol. 174 (2005) 1334).

However, in the trial described in J. Urology, Vol. 174 (2005) 1334, the adrenaline α receptor antagonist and the muscarinic receptor antagonist were directly administered to patients with prostatic hypertrophy accompanied with overactive bladder symptoms (urgency, pollakiuria, urinary incontinence and/or nocturia, and the like.) without preliminary selection of test patients by administering an adrenaline α receptor antagonist, and the like.

Thus, whilst the combination treatment with the alpha-adrenergic receptor antagonist and the muscarinic receptor antagonist further improved storage symptoms as compared to the treatment with the alpha-adrenergic receptor antagonist alone, the combination treatment antagonised the improving effect in voiding symptoms, namely, it exerted a negative effect on the voiding symptoms. This result could be expected. Propiverine however, did not affect the urinary flow rate and no acute AUR was observed.

As shown in Table 4 below, doxazosin alone improved the total I-PSS by 7.3 points while the combination of doxazosin and propiverine hydrochloride improved it by 7.4 points. With respect to the storage symptom score, doxazosin alone improved it by 2.9 points whereas the combination of doxazosin and propiveine hydrochlorice improved it by 3.7-3.8 points. Thus, with respect to the total I-PSS and storage symptom scores, improvement by the combination therapy with the muscarinic receptor antagonist was better than or similar to improvement by the therapy with the adrenaline α (alpha-andrenergic) receptor antagonist alone.

On the other hand, regarding the voiding symptom score, administration of doxazosin alone improved it by 4.5 points whereas the combination of doxazosin and propiverine hydrochloride improved it by only 3.7 points, indicating that the combination with the muscarinic receptor antagonist lowered the improving effect compared with the adrenaline α receptor antagonist alone.

Also, as regard to combination therapies with an adrenaline α receptor antagonist and a muscarinic receptor antagonist for improvement of lower urinary tract symptoms associated with prostatic hypertrophy, there is a report on a combination therapy using tamsulosin as an α receptor antagonist and tolterodine as a muscarinic receptor antagonist (JAMA, Vol. 296, No. 19 (2006) 2319).

JAMA, Vol. 296, No. 19 (2006) 2319 demonstrates that the combination therapy with tamsulosin and tolterodine significantly improved the total I-PSS compared to placebo, and that it also significantly improved, urgency, urination frequency in 24 hours and nocturnal urination frequency, each of which are storage symptom indices, compared to placebo. The reference describes the results of a twelve week clinical study in men with LUTS, characterized by an I-PPS-score of 12 or higher) and Overactive bladder (OAB), characterized by 8 or more micturitions/day and 3 or more urgency episodes per day.

JAMA, Vol. 296, No. 19 (2006) 2319 demonstrates that the combination therapy with tamsulosin and tolterodine significantly improved the total I-PSS compared to placebo, and that it also significantly improved, urgency, micturition frequency in 24 hours, and nocturnal micturition frequency each of which are the storage symptom indices, compared to placebo. However, as it appears the results for the combination are not significantly better than for the single drugs. Moreover, there is no description about its improving efficacy on voiding symptoms.

Tamsulosin or its salt is a known adrenaline α receptor antagonist (U.S. Pat. No. 4,703,063) and its chemical structure is shown below. Tamsulosin is also known as YM617. Tamsulosin or its pharmaceutically acceptable salt, an effective ingredient of the pharmaceutical composition of the present invention, is easily available by the methods described, for example, in U.S. Pat. No. 4,703,063, or by methods obvious to the person skilled in the art, or by modifying the same.

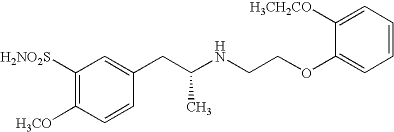

Solifenacin or its salt is known as a muscarinic receptor antagonist (U.S. Pat. No. 6,017,927) and its chemical structure is shown below. Solifenacin is also known as YM905. Solifenacin or its pharmaceutically acceptable salt, an effective ingredient of the pharmaceutical composition of the present invention, is easily available by the methods described, for example, in U.S. Pat. No. 6,017,927, or by methods obvious to the person skilled in the art, or by modifying the same.

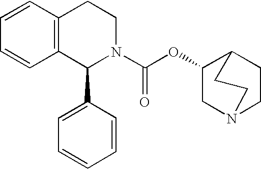

### SUMMARY OF THE INVENTION

It is an object of the invention to provide pharmaceutical compositions containing tamsulosin or its pharmaceutically acceptable salts and solifenacin or its pharmaceutically acceptable salts, particularly agents for improvement of lower urinary symptoms associated with prostatic hypertrophy.

In addition, combination use and combination therapy with tamsulosin, or its pharmaceutically acceptable salts, and solifenacin, or its pharmaceutically acceptable salts, for improvement of lower urinary tract symptoms associated with prostatic hypertrophy.

Further, the present invention provides a safe and effective treatment to improve the lower urinary tract symptoms associated with BPH and in particular to improve the storage symptoms, that have shown to be more bothersome especially in patients suffering from LUTS/BPH with a substantial storage component, without causing a substantial deterioration of the voiding symptoms.

The present inventors intensively studied improvement of lower urinary tract symptoms associated with prostatic hypertrophy, and as a result, discovered that the use of particularly tamsulosin, or its pharmaceutically acceptable salts, and solifenacin, or its pharmaceutically acceptable salts, exhibited unique improving effect on lower urinary tract symptoms associated with prostatic hypertrophy, and thus accomplished the present invention.

That is, the present invention relates to:

[1] A pharmaceutical composition, comprising (R)-5-(2-{[2-(2-ethoxyphenoxy)ethyl]amino}propyl-2-methoxybenzene-1-sulfonamide or its pharmaceutically acceptable salt, and (1S)-1-phenyl-1,2,3,4-tetrahydroisoquinoline-2-carboxylic acid (3R)-quinuclidin-3-yl ester or its pharmaceutically acceptable salt as active ingredients, together with a pharmaceutically acceptable excipient.
[2] The pharmaceutical composition according to [1], wherein (R)-5-(2-{[2-(2-ethoxyphenoxy)ethyl]amino}propyl-2-methoxybenzene-1-sulfonamide or its pharmaceutically acceptable salt is (R)-5-(2-{[2-(2-ethoxyphenoxy)ethyl]amino}propyl-2-methoxybenzene-1-sulfonamide hydrochloride.
[3] The pharmaceutical composition according to either [1] or [2], wherein (1S)-1-phenyl-1,2,3,4-tetrahydroisoquinoline-2-carboxylic acid (3R)-quinuclidin-3-yl ester or its pharmaceutically acceptable salt is (1S)-1-phenyl-1,2,3,4-tetrahydroisoquinoline-2-carboxylic acid (3R)-quinuclidin-3-yl ester succinate.
[4] A method of improving lower urinary tract symptoms associated with prostatic hypertrophy, comprising administering to a human male patient in need thereof a pharmaceutical composition according to either [1] or [2].
[5] A method of improving lower urinary tract symptoms associated with prostatic hypertrophy, comprising administering to a human male patient in need thereof a pharmaceutical composition according to [3].
[6] The method for improving lower urinary tract symptoms associated with prostatic hypertrophy according to [4], wherein the patient presents a total score of I-PSS is 8 or higher after administration of an adrenaline α receptor antagonist for 4 weeks or more.
[7] The method for improving lower urinary tract symptoms associated with prostatic hypertrophy according to [5], wherein the patient presents a total score of I-PSS is 8 or higher after administration of an adrenaline α receptor antagonist for 4 weeks or more.
[8] A pharmaceutical composition according to either [1] or [2], comprising 0.2 mg or 0.4 mg of (R)-5-(2-{[2-(2-ethoxyphenoxy)ethyl]amino}propyl-2-methoxybenzene-1-sulfonamide hydrochloride.
[9] A pharmaceutical composition according to [3], comprising 0.2 mg or 0.4 mg of (R)-5-(2-{[2-(2-ethoxyphenoxy)ethyl]amino}propyl-2-methoxybenzene-1-sulfonamide hydrochloride.
[10] A pharmaceutical composition according to either [1] or [2], comprising 2.5 mg or 5.0 mg of (1S)-1-phenyl-1,2,3,4-tetrahydroisoquinoline-2-carboxylic acid (3R)-quinuclidin-3-yl ester succinate.
[11] A pharmaceutical composition according to [3], comprising 2.5 mg or 5.0 mg of (1S)-1-phenyl-1,2,3,4-tetrahydroisoquinoline-2-carboxylic acid (3R)-quinuclidin-3-yl ester succinate.
[12] A pharmaceutical composition according to either [1] or [2], comprising 3 mg, 6 mg, or 9 mg of (1S)-1-phenyl-1,2,3,4-tetrahydroisoquinoline-2-carboxylic acid (3R)-quinuclidin-3-yl ester succinate.
[13] A pharmaceutical composition according to [3], comprising 3 mg, 6 mg, or 9 mg of (1S)-1-phenyl-1,2,3,4-tetrahydroisoquinoline-2-carboxylic acid (3R)-quinuclidin-3-yl ester succinate.
[14] A method of improving lower urinary tract symptoms associated with prostatic hypertrophy comprising administering to a human male patient in need thereof (R)-5-(2-{[2-(2-ethoxyphenoxy)ethyl]amino}propyl-2-methoxybenzene-1-sulfonamide hydrochloride and (1S)-1-phenyl-1,2,3,4-tetrahydroisoquinoline-2-carboxylic acid (3R)-quinuclidin-3-yl ester succinate either simultaneously or at a time interval.
[15] A method of treating lower urinary tract symptoms associated with prostatic hyperplasia with a substantial storage component, comprising the steps of simultaneously or sequentially administering to a human male patient in need thereof (R)-5-(2-{[2-(2-ethoxyphenoxy)ethyl]amino}propyl)-2-methoxybenzene-1-sulfonamide or a pharmaceutically acceptable salt thereof, and (1S)-1-phenyl-1,2,3,4-tetrahydroisoquinoline-2-carboxylic acid (3R)-quinuclidin-3-yl ester or a pharmaceutically acceptable salt thereof.
[16] A method of treating lower urinary tract symptoms associated with prostatic hyperplasia with a substantial storage component comprising the step of administering to a human male patient in need thereof the pharmaceutical composition according to [1].
[17] A method of treating lower urinary tract symptoms associated with prostatic hyperplasia with a substantial storage component comprising the step of administering to a human male patient in need thereof the pharmaceutical composition according to [2].
[18] A method of treating lower urinary tract symptoms associated with prostatic hyperplasia with a substantial storage component comprising the step of administering to a human male patient in need thereof the pharmaceutical composition according to [3].
[19] A method of treating lower urinary tract symptoms associated with prostatic hyperplasia with a substantial storage component comprising the step of administering to a human male patient in need thereof the pharmaceutical composition according to [8].
[20] A method of treating lower urinary tract symptoms associated with prostatic hyperplasia with a substantial storage component comprising the step of administering to a human male patient in need thereof the pharmaceutical composition according to [9].
[21] A method of treating lower urinary tract symptoms associated with prostatic hyperplasia with a substantial storage component comprising the step of administering to a human male patient in need thereof the pharmaceutical composition according to [10].
[22] A method of treating lower urinary tract symptoms associated with prostatic hyperplasia with a substantial storage component comprising the step of administering to a human male patient in need thereof the pharmaceutical composition according to [11].
[23] A method of treating lower urinary tract symptoms associated with prostatic hyperplasia with a substantial storage component comprising the step of administering to a human male patient in need thereof the pharmaceutical composition according to [12].
[24] A method of treating lower urinary tract symptoms associated with prostatic hyperplasia with a substantial storage component comprising the step of administering to a human male patient in need thereof the pharmaceutical composition according to [13].
[25] The method according to any one of [14]-[24], wherein said (R)-5-(2-{[2-(2-ethoxyphenoxy)ethyl]amino}propyl)-2-methoxybenzene-1-sulfonamide is provided in a modified-release formulation or a modified-release part of a combination composition.
[26] The method according to any one of [14]-[24], wherein said (1S)-1-phenyl-1,2,3,4-tetrahydroisoquinoline-2-carboxylic acid (3R)-quinuclidin-3-yl ester is provided in an immediate release formulation or in an immediate release part of a combination composition.
[27] A pharmaceutical composition, comprising 0.4 mg of (R)-5-(2-{[2-(2-ethoxyphenoxy)ethyl]amino}propyl)-2-methoxybenzene-1-sulfonamide hydrochloride and 6 mg or 9 mg of (1S)-1-phenyl-1,2,3,4-tetrahydroisoquinoline-2-carboxylic acid (3R)-quinuclidin-3-yl ester succinate, together with a pharmaceutically acceptable carrier.
[28] The method according to [16], wherein the lower urinary tract symptoms have been characterised by a total I-PSS score of 13 or higher and wherein the substantial storage component has been characterized as ≧8 micturitions/day and 1 urgency episode of PPIUS grade 3 or 4/day.
[29] The method according to [28], wherein the substantial storage symptoms have been characterised as ≧8 micturitions/day and ≧2 urgency episodes of grade 3 and 4/day.
[30] The method according either of [28] or [29], wherein the substantial storage symptoms have been characterized as ≧8 micturitions/day, ≧2 urgency episodes of grade 3 and 4/day and a Qmax of 4-12 mL/s.

Also, the present invention relates to a pharmaceutical composition for improvement of lower urinary tract symptoms associated with prostatic hypertrophy, which contains tamsulosin, or its pharmaceutically acceptable salt, and solifenacin, or its pharmaceutically acceptable salt, namely, an improving agent for lower urinary tract symptoms associated with prostatic hypertrophy, which contain tamsulosin, or its pharmaceutically acceptable salt, and solifenacin, or its pharmaceutically acceptable salt.

Additionally, the present invention relates to use of tamsulosin, or its pharmaceutically acceptable salt, and solifenacin, or its pharmaceutically acceptable salt, for manufacturing of a pharmaceutical composition for improvement of lower urinary tract symptoms associated with prostatic hypertrophy, and a method for improving lower urinary tract symptoms associated with prostatic hypertrophy comprising administering an effective dose of tamsulosin, or its pharmaceutically acceptable salt, and solifenacin, or its pharmaceutically acceptable salt to patients.

Also, the present invention provides the use of (R)-5-(2-{[2-(2-ethoxyphenoxy)ethyl]amino}propyl)-2-methoxybenzene-1-sulfonamide or a pharmaceutically acceptable salt thereof and (1S)-1-phenyl-1,2,3,4-tetrahydroisoquinoline-2-carboxylic acid (3R)-quinuclidin-3-yl ester or a pharmaceutically acceptable salt thereof for the preparation of a fixed-dose combination composition for improving storage symptoms in male patients having lower urinary tract symptoms associated with prostatic hyperplasia (LUTS/BPH) with a substantial storage component.

### DETAILED DESCRIPTION OF THE INVENTION

In the present description, “improvement of lower urinary tract symptoms associated with prostatic hypertrophy” means improvement of lower urinary tract symptoms in patients with benign prostatic hypertrophy, including (1) voiding symptoms such as weak stream, intermittency, delayed urination, and abdominal pressure urination, (2) storage symptoms such as pollakiuria, nocturia, and urgency, and (3) post-voiding symptoms such as emptying and post-urination dribbling.

Also, as the “adrenaline α receptor antagonists”, tamsulosin, doxazosin, alfuzosin, bunazosin, indolamine, naphthopidil, prazosin, terazosin, urapidil, and silodosin can be exemplified.

Tamsulosin or its pharmaceutically acceptable salt, an effective ingredient of a pharmaceutical composition of the present invention, is easily available by methods described, for example, in the aforementioned patent document 1, or by methods obvious to the person skilled in the art, or by their modifications.

Solifenacin or its pharmaceutically acceptable salt, an effective ingredient of a pharmaceutical composition of the present invention, is easily available by methods described, for example, in the aforementioned patent document 2, or by methods obvious to the person skilled in the art, or by their modifications.

Specific examples of the “pharmaceutically acceptable salts” in “tamsulosin or its pharmaceutically acceptable salt” and “solifenacin or its pharmaceutically acceptable salt” include addition salts with inorganic acids such as hydrochloric acid, hydrobromic acid, hydroiodic acid, sulfuric acid, nitric acid, and phosphoric acid, or with organic acids such as formic acid, acetic acid, propionic acid, oxalic acid, malonic acid, succinic acid, fumaric acid, maleic acid, lactic acid, malic acid, mandelic acid, tartaric acid, dibenzoyltartaric acid, ditoluoyltartaric acid, citric acid, methanesulfonic acid, ethanesulfonic acid, benzenesulfonic acid, p-toluenesulfonic acid, aspartic acid, and glutamic acid, and salts with inorganic bases such as sodium, potassium, magnesium, calcium, aluminium, or with organic bases such as methylamine, ethylamine, ethanolamine, lysine, and ornithine, and salts with various amino acids and amino acid derivatives such as acetylleucine, ammonium salts, and others.

Furthermore, the “pharmaceutically acceptable salts” in “tamsulosin or its pharmaceutically acceptable salt” and “solifenacin or its pharmaceutically acceptable salt” may be various hydrates, solvates and polymorphic crystalloids, which are all included as an active ingredient of the pharmaceutical composition of the present invention. The present invention also includes a pharmaceutical composition containing various radioactive or non-radioactive isotope-labeled compounds.

Preferably (R)-5-(2-{[2-(2-ethoxyphenoxy)ethyl]amino}propyl)-2-methoxybenzene-1-sulfonamide hydrochloride or tamsulosin hydrochloride is used. Preferably (1S)-1-phenyl-1,2,3,4-tetrahydroisoquinoline-2-carboxylic acid (3R)-quinuclidin-3-yl ester or solifenacin is used. The above-mentioned products, containing the first active ingredient, tamsulosin hydrochloride and the second active ingredient, solifenacin succinate, can also form part of a kit.

After extensive studies the present inventors found that the combined use of (R)-5-(2-{[2-(2-ethoxyphenoxy)ethyl]amino}propyl)-2-methoxybenzene-1-sulfonamide or a pharmaceutically acceptable salt thereof and (1S)-1-phenyl-1,2,3,4-tetrahydroisoquinoline-2-carboxylic acid (3R)-quinuclidin-3-yl ester or a pharmaceutically acceptable salt thereof demonstrated to be of benefit for the preparation of a medicament for improving storage symptoms in male patients having lower urinary tract symptoms associated with prostatic hyperplasia (LUTS/BPH) with substantial storage symptoms, as diagnosed by means of a total I-PPS-score (LUTS/BPH) and number of micturitions/day and urgency episodes/day (storage symptoms) respectively.

The present inventors further surprisingly found that the combined use of in particular tamsulosin hydrochloride and solifenacin succinate showed that the same combination had no or even an deteriorating effect on male patients having lower urinary tract symptoms associated with benign prostatic hyperplasia, without a substantial storage component.

Among pharmaceutical compositions or methods to improve lower urinary tract symptoms associated with prostatic hypertrophy in the present invention, a pharmaceutical composition to improve lower urinary tract symptoms or a method for improving lower urinary tract symptoms associated with prostatic hypertrophy in patients with prostatic hypertrophy whose total I-PSS scores are 8 or higher after administration of an adrenaline α receptor antagonist for 4 weeks or longer.

Among pharmaceutical compositions or methods to improve lower urinary tract symptoms associated with prostatic hypertrophy in the present invention, a pharmaceutical composition in which tamsulosin or its pharmaceutically acceptable salt is tamsulosin hydrochloride ((R)-5-(2-{[2-(2-ethoxyphenoxy)ethyl]amino}propyl-2-methoxybenzene-1-sulfonamide hydrochloride). This active ingredient is included in a single dose from 0.1 mg to 0.8 mg tamsulosin (or its salt, such as hydrochloride), or 0.1 mg, 0.2 mg, 0.4 mg, or 0.8 mg tamsulosin (or salt) in order to improve lower urinary tract symptoms associated with prostatic hypertrophy.

Advantageously the two active ingredients may be administered to patients suffering from LUTS/BPH with a substantial storage component in fixed dose combination compositions, wherein (R)-5-(2-{[2-(2-ethoxyphenoxy)ethyl]amino}propyl)-2-methoxybenzene-1-sulfonamide hydrochloride or tamsulosin hydrochloride is used in an amount of 0.4 mg and (1S)-1-phenyl-1,2,3,4-tetrahydroisoquinoline-2-carboxylic acid (3R)-quinuclidin-3-yl ester succinate or solifenacin succinate is used in an amount of 3 mg, 6 mg or 9 mg, preferably 6 mg or 9 mg.

Among pharmaceutical compositions or methods to improve lower urinary tract symptoms associated with prostatic hypertrophy in the present invention, a pharmaceutical composition in which solifenacin or its pharmaceutically acceptable salt is solifenacin succinate ((1S)-1-phenyl-1,2,3,4-tetrahydroisoquinoline-2-carboxylic acid (3R)-quinuclidin-3-yl ester succinate). This active ingredient is included in a single dose at from 2.5 mg to 10 mg solifenacin (or its salt, such as succinate), or 2.5 mg, 3 mg, 5 mg, 6 mg, 9 mg, or mg solifenacin (or its salt) in order to improve lower urinary tract symptoms in patients with prostatic hypertrophy, or a method to improve lower urinary tract symptoms associated with prostatic hypertrophy.

The pharmaceutical composition of the present invention can be prepared by usually employed methods with tamsulosin or its pharmaceutically acceptable salt and solifenacin or its pharmaceutically acceptable salt along with carriers, excipients, or other additives for usual drug formulation. Administration can be performed orally in the forms of tablets, pills, capsules, granules, powders, liquids and the like, or parenterally in the forms of intra-articular, intravenous, or intramuscular injections, suppositories, ophthalmic solutions, ointments, percutaneous liquids, ointments, transdermal patches, transmucosal liquids, transmucosal patches, or inhalants. Oral administration can be cited as a preferred aspect.

Normally the active ingredients are included in a modified-release formulation, in the form of coated granules in a capsule (commercially available as OMNIC® or HARNAL® capsules) or in the form of coated matrix tablets (commercially available as OMNIC OCAS® tablets). Alternatively, the active ingredient can be incorporated in a modified-release part of a combination composition. The active ingredient may also be incorporated in a coated immediate release dosage form (commercially available as VESICARE® tablet), or in an immediate release part of a combination dosage-form.

As compositions for oral administration, tablets, pills, capsules, powders, granules, liquids and the like are used. In these solid compositions, the effective ingredient is mixed with at least one of inert carriers, excipients or other additives and the like for usual drug formulation. The composition may follow conventional methods to contain inert additives such as lubricants, disintegrators, stabilizers, or solubilizers. If necessary, tablets and pills may be covered with sugar or gastric- or enteric-coating films. Administration can also be performed parenterally in the forms of intra-articular, intravenous, or intramuscular injections, suppositories, ophthalmic solutions, ointments, percutaneous liquids, ointments, transdermal patches, transmucosal liquids, transmucosal patches, or inhalants. Oral administration is identified as a preferred embodiment.

Liquid compositions for oral administration contain pharmaceutically acceptable emulsifiers, solubilizers, suspensions, syrups, or elixirs, and the like. They also contain generally employed inert diluents such as purified water or ethanol. These liquid compositions may also, in addition to inert diluents, adjuvants such as solubilizers, humidifiers or suspensions, or sweetening agents, flavoring agents, aromatic agents, or antiseptic agents.

Injections for parenteral administration contain aseptic aqueous or non-aqueous solubilizers, suspensions, or emulsifiers. Aqueous solvents include, for example, distilled water for injection or physiological saline. As non-aqueous solvents, there are propylene glycol, polyethylene glycol, vegetable oils such as olive oil, alcohols such as ethanol, and Polysorbate 80 (Japanese Pharmacopea), and the like. Such compositions may further contain isotonizing agents, antiseptic agents, humidifying agents, emulsifying agents, dispersing agents, stabilizing agents or solubilizing agents. These may be sterilized by filtration through, for example, a bacterium-retaining filter, addition of a sterilizer, or irradiation. These may also be prepared by preparing solid compositions aseptically and then solubilizing or suspending them in sterilized water or a sterilized solvent for injection.

Topical preparations include ointments, plasters, creams, jellies, epithems, nebulas, lotions, ophthalmic solutions or ointments, and the like. They contain generally employed ointment bases, lotion bases, aqueous or non-aqueous liquids, suspending agents, emulsifying agents, and the like. The ointment and lotion bases include, for example, polyethylene glycol, propylene glycol, white petrolatum, bleached beeswax, polyoxyethylene-hardened caster oil, glycerin monostearate, stearyl alcohol, cetyl alcohol, lauromacrogol, sorbitan sesquioleate, and the like.

Transmucosal agents such as inhalants or transnasal agents utilize solids, liquids, or semi-liquids, and they can be prepared by conventional methods. For example, known excipients, or further, pH adjusters, antiseptics, surfactants, lubricants, stabilizers, or thickeners may properly be added. Drug administration may be aided by appropriate devices for inhalation or insufflation. For example, by using known devices such as pre-measured inhalation devices or nebulizers, a compound can be administered alone or as a formulated mixture in a powder, or in combination with pharmaceutically acceptable carriers in a solution or suspension. Dry-powder inhalers may be for single or multiple dosage of dry powder or powder-containing capsules, or may be assisted by a pressurized aerosol sprayer utilizing appropriate gases such as chlorofluoroalkanes, hydrofluoroalkanes, or carbon dioxide as propelling agents.

Combination administration in the present invention may be performed by administering two drugs simultaneously, one immediately after the other, or with a desired time interval between them. When administered simultaneously, a combination drug that is a single drug composition in which both effective ingredients are contained, or two separate formulations each of which contains each effective ingredient may be administered.

The combined use of the two active ingredients, tamsulosin hydrochloride and solifenacin succinate is particularly useful for improving the symptoms of patients having Lower Urinary Tract Symptoms, associated with Benign Prostatic Hyperplasia (LUTS/BPH) wherein the severity of the lower urinary tract symptoms has been characterised by a total I-PSS score of 13 or higher and wherein the substantial storage symptoms have been characterized as ≧8 micturitions/day and ≧1, preferably ≧2 urgency episodes/day. More particularly the patients have urgency episodes, that are characterized as grade 3 or 4 according to the PPIUS scale.

More particularly the combined use of the first active ingredient and the second active ingredient in accordance with the above has been shown to be very useful for improving the lower urinary tract symptoms associated with BPH in male patients, the symptoms having been characterised by a total score of I-PSS of 13 or higher and wherein the substantial storage symptoms have been characterized as ≧8 micturitions/day, ≧2 urgency episodes of grade 3 and 4/day and a Qmax of 4-12 mL/s.

Efficacy of the combination therapy with the effective ingredients of the pharmaceutical compositions according to the present invention has been clinically confirmed by the following examples.

### Example 1

Male human patients with prostatic hypertrophy who still had symptoms of overactive bladder (urgency, pollakiuria, urinary incontinence and/or nocturia, and the like.) after treatment with an α receptor antagonist for four weeks or longer were allocated for administration to two treatment groups: (1) tamsulosin hydrochloride 0.2 mg (hereinafter referred to as “Single Group”), and (2) tamsulosin hydrochloride 0.2 mg and solifenacin succinate 2.5 mg (hereinafter referred to as “Combination Group”), and then their symptoms were evaluated by I-PSS after four weeks of treatment. The results are shown in Table 1 below. In Table 1 since the score of I-PSS becomes greater as the symptom worsens, greater minus change in scores indicates greater efficacy for the symptoms.

TABLE 1

| | | | After | |
|---|---|---|---|---|
| | | Before | treatment of | |
| | | treatment | 4 weeks | Change |
| Total scores of | Single | 11.6 | 10.8 | −0.8 |
| I-PSS | Combination | 13.9 | 11.2 | −2.7 |
| Scores of storage | Single | 6.5 | 5.8 | −0.7 |
| symptoms | Combination | 7.2 | 5.5 | −1.7 |
| (1) (3) (5) (6) | | | | |
| Scores of voiding | Single | 4.2 | 4.2 | 0.0 |
| symptoms | Combination | 5.3 | 4.6 | −0.6 |
| (2) (4) (7) | | | | |

As shown in Table 1, the combination therapy with tamsulosin hydrochloride and solifenacin succinate showed improvement effect on the total I-PSS and storage symptom scores in comparison with the single therapy with tamsulosin hydrochloride, and it gave no effect on the voiding symptom score unexpectedly, or it even gave a further improving tendency in comparison with the single therapy with tamsulosin hydrochloride. Namely, it is clear that the combination therapy with tamsulosin hydrochloride and solifenacin succinate did not worsen the voiding symptom index in comparison with the single therapy with tamsulosin hydrochloride.

### Example 2

In new male human patients and those with prostatic hypertrophy and remaining overactive bladder symptoms after treatment with tamsulosin hydrochloride for 4 weeks or longer, the following treatments were performed:

(1) New patients were treated with 0.2 mg tamsulosin hydrochloride for four weeks.
(2) Out of the new patients who were treated with 0.2 mg tamsulosin hydrochloride for four weeks, those who still required treatment for their overactive bladder symptoms, and those patients who received tamsulosin hydrochloride for four weeks or longer but still showed overactive bladder symptoms were treated with 0.2 mg tamsulosin hydrochloride and 2.5 mg solifenacin succinate for eight weeks.
(3) For those who received solifenacin succinate for four weeks without obtaining sufficient clinical relief, the dose of solifenacin succinate was raised to 5.0 mg for the remaining four weeks according to the patients' direction.

The patients' symptoms were evaluated by I-PSS before and after the treatment with solifenacin succinate for eight weeks. The results are shown below in Table 2.

TABLE 2

| | Before | After 8 weeks | |
|---|---|---|---|
| | combination | of combination | |
| | therapy | therapy | Change |
| Total scores of | 14.5 | 10.6 | −3.9 |
| I-PSS | | | |
| Scores of storage | 7.9 | 4.8 | −3.1 |
| symptoms | | | |
| (1) (3) (5) (6) | | | |
| Scores of voiding | 5.1 | 5.0 | −0.1 |
| symptoms | | | |
| (2) (4) (7) | | | |

As shown in Table 2, the combination therapy with tamsulosin hydrochloride and solifenacin succinate showed improving efficacy on the total I-PSS and storage symptom scores in comparison with the single therapy with tamsulosin hydrochloride, and furthermore it gave no effect on the voiding symptom score unexpectedly in comparison with the single therapy with tamsulosin hydrochloride. Namely, it is clear that the combination therapy with tamsulosin hydrochloride and solifenacin succinate did not worsen the voiding symptom index in comparison with the single therapy with tamsulosin hydrochloride.

### Example 3

Similar evaluation can be performed similarly except that the dose of tamsulosin hydrochloride is changed to 0.2 mg or 0.4 mg, and the dose of solifenacin succinate is changed to 2.5 mg, 5 mg, or 10 mg, or 3 mg, 6 mg, or 9 mg from the dosages in Examples 1 and 2.

Contrary to the notion that while the combination therapy with an adrenaline α receptor antagonist and a muscarinic receptor antagonist further improves storage symptoms in comparison with the single therapy with an adrenaline α receptor antagonist, it inhibits the improvement, namely, exerts a negative effect on voiding symptoms, the above results have unexpectedly demonstrated that the unique combination of tamsulosin or its pharmaceutically acceptable salt with solifenacin or its pharmaceutically acceptable salt neither interfere with nor influence the improving effect of tamsulosin on voiding symptoms in comparison with the improving effect of the single therapy with tamsulosin or its pharmaceutically acceptable salt alone on voiding symptoms, and that it exerts improving effect on the total I-PSS and storage symptom scores.

### Example 4

In a single-blind, two-week placebo run-in period followed by a randomized, double-blind, parallel group, placebo-controlled, twelve-week treatment period multi-center dose-ranging study, male patients having LUTS associated with BPH who had voiding symptoms (including incomplete emptying of the bladder, intermittency, weak stream or hesitancy) and storage symptoms (including frequency, urgency or nocturia) for ≧3 months, the severity of their symptoms being characterised by a total International Prostate Symptom Score (I-PSS) of ≧13 and a maximum urinary flow rate of ≧4.0 mL/s and ≦15.0 mL/s with a voided volume ≧120 mL, were randomized to one of the following eight treatments:
placebo,
monotherapy of 0.4 mg tamsulosin hydrochloride in a modified-release OCAS formulation (commercially available as OMNIC OCAS® tablets),
monotherapy of 3, 6, or 9 mg respectively of solifenacin succinate in an immediate release tablet formulation or
combination therapy of 0.4 mg of tamsulosin hydrochloride in a modified-release OCAS formulation (commercially available as OMNIC OCAS® tablets) in combination with 3, 6, or 9 mg respectively of solifenacin succinate in an immediate release tablet formulation.

Throughout the placebo run-in period, subjects took two placebo tablets once daily. Throughout the twelve-week double-blind treatment period, subjects took two tablets once daily (tamsulosin hydrochloride OCAS 0.4 mg or placebo and solifenacin succinate 3, 6 or 9 mg or placebo). Study medication was taken orally in the morning with or without food. Medication was taken with a glass of water and was swallowed whole.

Analyses were carried out on subpopulations based on the severity of baseline storage symptoms. The subpopulations investigated were Storage symptoms subgroups 1, 2 and 3, with a daily micturition frequency ≧8 and ≧1, 2 or 3 urgency episodes of grade 3 and 4 per day (PPIUS), respectively. The fourth subgroup was the Limited storage symptoms subgroup, whose baseline symptoms did not meet the criteria for Storage symptoms subgroup 1. Total urgency score, the mean sum of all urgency grades (PPIUS) per day was added as a new parameter.

### Summary of I-PSS Data:

Opposite treatment effects were seen between the Limited storage symptoms subgroup and the Storage symptoms subgroups. The present inventors determined:
When using higher doses of solifenacin in combination with tamsulosin OCAS alone, a deterioration was seen in the Limited storage symptoms subgroup for total I-PSS score, whereas the Storage symptoms subgroups 1, 2 and 3 showed a non-statistically significant trend to improvement.
A deterioration in the I-PSS voiding scores was seen for all subgroups, when using combination treatment versus tamsulosin monotherapy. The only statistically significant slope with increasing dosages of solifenacin and in the presence of tamsulosin was in the Limited storage symptoms subgroup (p=0.0006).
For I-PSS storage scores, all 3 subgroups with Storage symptoms showed a statistically significant improvement in the 3 different combination treatment arms versus monotherapy and in dose response (p=0.0016, p<0.0001, p=0.0006 respectively), while the Limited storage symptoms subgroup showed a not statistically significant deteriorating trend.
All 3 subgroups with Storage symptoms showed a statistically significant trend to improvement in the I-PSS QoL score (p=0.0094, p=0.0008 and p=0.0106 respectively) with solifenacin-tamsulosin OCAS combination therapy compared to tamsulosin OCAS alone, while the Limited storage symptoms subgroup showed a statistically significant deteriorating trend (see Table 3).

TABLE 3
Parametric modeling results: change from baseline to endpoint in I-PSS scores showing
estimated difference between solifenacin - tamsulosin OCAS combination versus tamsulosin
OCAS in subpopulations based on severity of baseline storage symptoms (FAS)

| | Sol | | | Limited storage | | Storage Symptoms | | | | | |
|---|---|---|---|---|---|---|---|---|---|---|---|
| Parameter | dose | All subjects | | Symptoms | | Subgroup 1 | | Subgroup 2 | | Subgroup 3 | |
| I-PSS Total | n | 704 | | 348 | | 344 | | 282 | | 225 | |
| | 3 mg | 0.14 | | 0.85 | | −0.65 | | −0.81 | | −0.59 | |
| | 6 mg | −0.01 | | 0.80 | | −0.63 | | −1.13 | | −1.44 | |
| | 9 mg | 1.00 | | 2.59 | *0.0013 | −0.87 | | −1.72 | | −1.56 | |
| | slope | | | | ↓0.0026 | | | | | | |
| I-PSS | n | 704 | | 348 | | 344 | | 282 | | 225 | |
| Voiding | 3 mg | 0.49 | | 0.78 | | 0.06 | | 0.1 | | 0.47 | |
| | 6 mg | 0.40 | | 0.57 | | 0.35 | | 0.17 | | −0.01 | |
| | 9 mg | 1.33 | *0.0009 | 2.18 | *0.0002 | 0.25 | | −0.12 | | 0.05 | |
| | slope | | ↓0.0022 | | ↓0.0006 | | | | | | |
| I-PSS | n | 704 | | 348 | | 344 | | 282 | | 225 | |
| Storage | 3 mg | −0.32 | | 0.15 | | −0.7 | *0.0484 | −0.88 | *0.0286 | −0.96 | *0.0389 |
| | 6 mg | −0.41 | | 0.24 | | −0.98 | *0.0071 | −1.28 | *0.0022 | −1.38 | *0.0045 |
| | 9 mg | −0.33 | | 0.44 | | −1.12 | *0.0021 | −1.6 | *0.0001 | −1.59 | *0.0008 |
| | slope | | | | | | ↑0.0016 | | ↑<0.0001 | | ↑0.0006 |
| I-PSS- QoL | n | 703 | | 343 | | 348 | | 281 | | 224 | |
| | 3 mg | −0.13 | | 0.17 | | −0.45 | *0.0171 | −0.5 | *0.0153 | −0.49 | *0.0380 |
| | 6 mg | −0.01 | | 0.31 | | −0.31 | | −0.5 | *0.0189 | −0.38 | |
| | 9 mg | −0.06 | | 0.40 | *0.0277 | −0.58 | *0.0025 | −0.77 | *0.0004 | −0.70 | *0.0043 |
| | slope | | | | ↓0.0196 | | ↓0.0094 | | ↑0.0008 | | ↑0.0106 |

Sol dose = solifenacin dosage in mg in combination with tamsulosin OCAS 0.4 mg; Difference on left and significant p-value on right of column; n = total number of subjects in the subgroup with relevant data at any dose of solifenacin;
*statistically significant (p < 0.05);
↑statistically significant slope showing improvement;
↓statistically significant slope showing deterioration (p < 0.05)

The micturition data may be summarized as follows:
Storage symptoms subgroups 2 and 3 showed a statistically significant dose-related improvement on all parameters, when adding solifenacin to tamsulosin OCAS versus tamsulosin OCAS alone. The solifenacin 6 mg—tamsulosin OCAS combination group showed a statistically significant improvement on all parameters, and the solifenacin 9 mg—tamsulosin OCAS combination group on total urgency score, frequency and voided volume (see Table 4.)
A statistically significant dose-related improvement for urgency episodes of grade 3 and 4 (PPIUS) was seen in Storage symptoms subgroups 2 and 3 (p=0.0128 and p=0.0241, respectively), when using solifenacin—tamsulosin OCAS combination treatment versus tamsulosin alone. There was also a statistically significant improvement for the 6 mg solifenacin—tamsulosin OCAS combination arm versus tamsulosin monotherapy in the 3 subgroups with Storage symptoms.
For total urgency score there was a statistically significant improvement for the three subgroups with Storage symptoms (p=0.0013, p=0.0004 and p=0.0038, respectively), and a statistically significant improvement for both the 6 and 9 mg solifenacin—tamsulosin OCAS combination arms versus tamsulosin OCAS monotherapy in the 3 subgroups with Storage symptoms.
The three subgroups with Storage symptoms showed a statistically significant dose-related improvement for frequency (p=0.0004, p=0.0003 and p=0.0032, respectively), and all three dosages of the solifenacin—tamsulosin OCAS combination arms versus tamsulosin OCAS monotherapy showed a statistically significant improvement.
When adding solifenacin to tamsulosin OCAS voided volume showed a statistically significant dose-related improvement for all four subgroups versus tamsulosin OCAS monotherapy (p<0.0001, p=0.0003, p=0.0042 and p=0.0067, respectively), and also a statistically significant improvement for the 9 mg solifenacin—tamsulosin OCAS combination arm versus tamsulosin OCAS monotherapy in all four subgroups.

The positive results in Storage symptoms subgroup 1 were mainly driven by the positive results in Storage symptoms subgroup 2.

TABLE 4
Parametric modeling results: change from baseline to endpoint in micturition diary data
showing estimated difference between solifenacin - tamsulosin OCAS combination versus tamsulosin
OCAS in subpopulations based on severity of baseline storage symptoms (FAS)

| | Sol | | | Limited storage | | Storage Symptoms | | | | | |
|---|---|---|---|---|---|---|---|---|---|---|---|
| Parameter | dose | All subjects | | Symptoms | | Subgroup 1 | | Subgroup 2 | | Subgroup 3 | |
| Urgency | n | 465 | | 124 | | 341 | | 280 | | 224 | |
| 3/4 | 3 mg | −0.1 | | −0.14 | | −0.15 | | −0.06 | | 0.07 | |
| | 6 mg | −0.83 | *0.0075 | −0.24 | | −0.95 | *0.0165 | −1.14 | *0.0138 | −1.47 | *0.0078 |
| | 9 mg | −0.24 | | 0.24 | | −0.47 | | −0.81 | | −0.78 | |
| | slope | | | | | | | | ↑0.0128 | | ↑0.0241 |
| Total | n | 678 | | 335 | | 341 | | 280 | | 224 | |
| Urgency | 3 mg | −1.18 | *0.0055 | −1.15 | | −1.65 | | −1.79 | | −1.57 | |
| score | 6 mg | −1.96 | | −0.53 | | −3.36 | *0.0028 | −3.93 | *0.0026 | −4.57 | *0.0029 |
| | 9 mg | −1.24 | | 0.18 | | −3.2 | *0.0042 | −4.08 | *0.0017 | −3.57 | *0.0166 |
| | slope | | ↑0.0437 | | | | ↑0.0013 | | ↑0.0004 | | ↑0.0038 |
| Frequency | n | 699 | | 347 | | 342 | | 280 | | 224 | |
| | 3 mg | −0.49 | *0.0131 | −0.1 | | −0.88 | *0.0040 | −0.9 | *0.0112 | −1.01 | *0.0129 |
| | 6 mg | −0.66 | *0.0009 | −0.35 | | −0.94 | *0.0028 | −0.96 | *0.0086 | −1.03 | *0.0163 |
| | 9 mg | −0.64 | *0.0012 | −0.2 | | −1.16 | *0.0002 | −1.4 | *0.0001 | −1.3 | *0.0019 |
| | slope | | ↑0.0008 | | | | ↑0.0004 | | ↑0.0003 | | ↑0.0032 |
| Voided | n | 699 | | 347 | | 342 | | 280 | | 224 | |
| volume | 3 mg | 6.3 | | 2.23 | | 10.06 | | 6.46 | | 5.90 | |
| | 6 mg | 18.3 | *<0.0001 | 21.49 | *0.0010 | 14.26 | *0.0341 | 10.18 | | 11.93 | |
| | 9 mg | | *<0.0001 | 26.11 | *<0.0001 | 24.45 | *0.0003 | 21.51 | *0.0048 | 21.37 | *0.0097 |
| | slope | 24.4 | ↑<0.0001 | | ↑<0.0001 | | ↑0.0003 | | ↑0.0042 | | ↑0.0067 |

Sol dose = solifenacin dosage in mg in combination with tamsulosin OCAS 0.4 mg; Difference on left and significant p-value on right of column; n = total number of subjects in the subgroup with relevant data at any dose of solifenacin;
*statistically significant (p < 0.05);
↑statistically significant slope showing improvement;
↓statistically significant slope showing deterioration (p < 0.05)

1. A pharmaceutical composition, comprising (R)-5-(2-{[2-(2-ethoxyphenoxy)ethyl]amino}propyl-2-methoxybenzene-1-sulfonamide or its pharmaceutically acceptable salt, and (1S)-1-phenyl-1,2,3,4-tetrahydroisoquinoline-2-carboxylic acid (3R)-quinuclidin-3-yl ester or its pharmaceutically acceptable salt as active ingredients, together with a pharmaceutically acceptable excipient.

2. The pharmaceutical composition according to claim 1, wherein (R)-5-(2-{[2-(2-ethoxyphenoxy)ethyl]amino}propyl-2-methoxybenzene-1-sulfonamide or its pharmaceutically acceptable salt is (R)-5-(2-{[2-(2-ethoxyphenoxy)ethyl]amino}propyl-2-methoxybenzene-1-sulfonamide hydrochloride.

3. The pharmaceutical composition according to either of claim 1 or 2, wherein (1S)-1-phenyl-1,2,3,4-tetrahydroisoquinoline-2-carboxylic acid (3R)-quinuclidin-3-yl ester or its pharmaceutically acceptable salt is (1S)-1-phenyl-1,2,3,4-tetrahydroisoquinoline-2-carboxylic acid (3R)-quinuclidin-3-yl ester succinate.

4. A method of improving lower urinary tract symptoms associated with prostatic hypertrophy, comprising administering to a human male patient in need thereof a pharmaceutical composition according to either of claim 1 or 2.

5. A method of improving lower urinary tract symptoms associated with prostatic hypertrophy, comprising administering to a human male patient in need thereof a pharmaceutical composition according to claim 3.

6. The method for improving lower urinary tract symptoms associated with prostatic hypertrophy according to claim 4, wherein the patient presents a total score of I-PSS is 8 or higher after administration of an adrenaline α receptor antagonist for 4 weeks or more.

7. The method for improving lower urinary tract symptoms associated with prostatic hypertrophy according to claim 5, wherein the patient presents a total score of I-PSS is 8 or higher after administration of an adrenaline α receptor antagonist for 4 weeks or more.

8. A pharmaceutical composition according to either of claim 1 or 2, comprising 0.2 mg or 0.4 mg of (R)-5-(2-{[2-(2-ethoxyphenoxy)ethyl]amino}propyl-2-methoxybenzene-1-sulfonamide hydrochloride.

9. A pharmaceutical composition according to claim 3, comprising 0.2 mg or 0.4 mg of (R)-5-(2-{[2-(2-ethoxyphenoxy)ethyl]amino}propyl-2-methoxybenzene-1-sulfonamide hydrochloride.

10. A pharmaceutical composition according to either of claim 1 or 2, comprising 2.5 mg or 5.0 mg of (1S)-1-phenyl-1,2,3,4-tetrahydroisoquinoline-2-carboxylic acid (3R)-quinuclidin-3-yl ester succinate.

11. A pharmaceutical composition according to claim 3, comprising 2.5 mg or 5.0 mg of (1S)-1-phenyl-1,2,3,4-tetrahydroisoquinoline-2-carboxylic acid (3R)-quinuclidin-3-yl ester succinate.

12. A pharmaceutical composition according to either of claim 1 or 2, comprising 3 mg, 6 mg, or 9 mg of (1S)-1-phenyl-1,2,3,4-tetrahydroisoquinoline-2-carboxylic acid (3R)-quinuclidin-3-yl ester succinate.

13. A pharmaceutical composition according to claim 3, comprising 3 mg, 6 mg, or 9 mg of (1S)-1-phenyl-1,2,3,4-tetrahydroisoquinoline-2-carboxylic acid (3R)-quinuclidin-3-yl ester succinate.

14. A method of improving lower urinary tract symptoms associated with prostatic hypertrophy comprising administering to a human male patient in need thereof (R)-5-(2-{[2-(2-ethoxyphenoxy)ethyl]amino}propyl-2-methoxybenzene-1-sulfonamide hydrochloride and (1S)-1-phenyl-1,2,3,4-tetrahydroisoquinoline-2-carboxylic acid (3R)-quinuclidin-3-yl ester succinate either simultaneously or at a time interval.

15. A method of treating lower urinary tract symptoms associated with prostatic hyperplasia with a substantial storage component, comprising the steps of simultaneously or sequentially administering to a human male patient in need thereof (R)-5-(2-{[2-(2-ethoxyphenoxy)ethyl]amino}propyl)-2-methoxybenzene-1-sulfonamide or a pharmaceutically acceptable salt thereof, and (1S)-1-phenyl-1,2,3,4-tetrahydroisoquinoline-2-carboxylic acid (3R)-quinuclidin-3-yl ester or a pharmaceutically acceptable salt thereof.

16. A method of treating lower urinary tract symptoms associated with prostatic hyperplasia with a substantial storage component comprising the step of administering to a human male patient in need thereof the pharmaceutical composition according to claim 1.

17. A method of treating lower urinary tract symptoms associated with prostatic hyperplasia with a substantial storage component comprising the step of administering to a human male patient in need thereof the pharmaceutical composition according to claim 2.

18. A method of treating lower urinary tract symptoms associated with prostatic hyperplasia with a substantial storage component comprising the step of administering to a human male patient in need thereof the pharmaceutical composition according to claim 3.

19. A method of treating lower urinary tract symptoms associated with prostatic hyperplasia with a substantial storage component comprising the step of administering to a human male patient in need thereof the pharmaceutical composition according to claim 8.

20. A method of treating lower urinary tract symptoms associated with prostatic hyperplasia with a substantial storage component comprising the step of administering to a human male patient in need thereof the pharmaceutical composition according to claim 9.

21. A method of treating lower urinary tract symptoms associated with prostatic hyperplasia with a substantial storage component comprising the step of administering to a human male patient in need thereof the pharmaceutical composition according to claim 10.

22. A method of treating lower urinary tract symptoms associated with prostatic hyperplasia with a substantial storage component comprising the step of administering to a human male patient in need thereof the pharmaceutical composition according to claim 11.

23. A method of treating lower urinary tract symptoms associated with prostatic hyperplasia with a substantial storage component comprising the step of administering to a human male patient in need thereof the pharmaceutical composition according to claim 12.

24. A method of treating lower urinary tract symptoms associated with prostatic hyperplasia with a substantial storage component comprising the step of administering to a human male patient in need thereof the pharmaceutical composition according to claim 13.

25. The method according to any one of claims 14-17, wherein said (R)-5-(2-{[2-(2-ethoxyphenoxy)ethyl]amino}propyl)-2-methoxybenzene-1-sulfonamide is provided in a modified-release formulation or a modified-release part of a combination composition.

26. The method according to any one of claims 14-17, wherein said (1S)-1-phenyl-1,2,3,4-tetrahydroisoquinoline-2-carboxylic acid (3R)-quinuclidin-3-yl ester is provided in an immediate release formulation or in an immediate release part of a combination composition.

27. A pharmaceutical composition, comprising 0.4 mg of (R)-5-(2-{[2-(2-ethoxyphenoxy)ethyl]amino}propyl)-2-methoxybenzene-1-sulfonamide hydrochloride and 6 mg or 9 mg of (1S)-1-phenyl-1,2,3,4-tetrahydroisoquinoline-2-carboxylic acid (3R)-quinuclidin-3-yl ester succinate, together with a pharmaceutically acceptable carrier.

28. The method according to claim 16, wherein the lower urinary tract symptoms have been characterised by a total I-PSS score of 13 or higher and wherein the substantial storage component has been characterized as 8 micturitions/day and 1 urgency episode of PPIUS grade 3 or 4/day.

29. The method according to claim 28, wherein the substantial storage symptoms have been characterised as ≧8 micturitions/day and ≧2 urgency episodes of grade 3 and 4/day.

30. The method according either of claim 28 or 29, wherein the substantial storage symptoms have been characterized as ≧8 micturitions/day, ≧2 urgency episodes of grade 3 and 4/day and a Qmax of 4-12 mL/s.
